(19) United States
(12) Patent Application Publication
(10) Pub. No.: US 2026/0198974 A1
(43) Pub. Date: Jul. 16, 2026

(54) TULIP SIDECAR DEVICES, SYSTEMS, AND METHODS

(71) Applicants: Juan Felix Ronderos, Mobile, AL (US); Chris Danek, San Carlos, CA (US); Andres Eduardo Guitierrez Hernandez, El Paso, TX (US)

(72) Inventors: Juan Felix Ronderos, Mobile, AL (US); Chris Danek, San Carlos, CA (US); Andres Eduardo Guitierrez Hernandez, El Paso, TX (US)

(21) Appl. No.: 19/103,787

(22) PCT Filed: Aug. 16, 2023
(86) PCT No.: PCT/US2023/072287

§ 371 Date: Feb. 13, 2025

Related U.S. Application Data
(60) Provisional application No. 63/398,398, filed on Aug. 16, 2022.
(60) Provisional application No. 63/488,592, filed on Mar. 6, 2023.

(51) Int. Cl. A61B 17/70; A61B 17/00

(52) U.S. Cl. CPC A61B 17/7052; A61B 17/7032; A61B 2017/00486; A61B 2017/00526

(57) ABSTRACT

Devices and systems for securing an adaptor device to a tulip structure on a pedicle screw is disclosed. The devices and systems may be configured to accept a spinal rod, and may include one or more of an adaptor, a set screw, a lock nut, and a tool, useful in securing the adaptor to the tulip and/or spinal rods to the adaptor and/or tulip. Additional embodiments of the devices and systems may include a tulip configured to engage the adaptor and other system components and/or an adaptor-to-adaptor device for connecting to the adaptor.

### CROSS-REFERENCE TO RELATED APPLICATION

This application claims the filing benefit of U.S. Provisional Application No. 63/398,398, filed Aug. 16, 2022, and 63/488,592, filed Mar. 6, 2023. This application is incorporated by reference herein in its entirety and for all purposes.

### FIELD

The disclosed processes, methods, and systems are directed to spinal fixation hardware.

### BACKGROUND

Pedicle anchor screws typically include, or can be fitted with, a structure having a slotted, hemispherical opening for receiving and securing a spinal rod. In many cases, these slotted structures, referred to as ‘tulips’ due to their resemblance of the flower, may allow the slot to be rotated and angled relative to the body of the pedicle screw. This polyaxial capability is useful for receiving the spinal rod. The tulip's slotted opening includes internal threads configured for receiving a set screw. The set screw is used to lock, anchor, or fix the rod securely within the tulip.

Many procedures may benefit from placement of additional spinal rods. In these cases, additional rods are secured, via a connector, to the existing rod. Such rod-to-rod connections may be problematic. For example, because the rods are cylindrical, it may be difficult to prevent rotational movement of the second rod, or the existing rod may be fractured or broken, which may affect stability.

What is needed are devices and systems for installation of additional spinal rods, where rod-to-rod attachments are avoided, and the additional rod is securely held.

### SUMMARY

Disclosed herein are methods and systems for securing one or more spinal rods to a pedicle screw tulip. In various aspects, a device for engaging a tulip and spinal rod is disclosed, the device including a channel, wherein the channel may be configured to accept and hold a spinal rod, and a yoke, wherein the yoke may be configured to engage a tulip. The device may include an adaptor set screw for securing the spinal rod in the channel, wherein the adaptor set screw may include threading corresponding to threading within the channel. In some embodiments, the adaptor set screw may include a threaded post and/or a recess for receiving a tool. The device may also include a tulip set screw for securing a spinal rod in the tulip, which may include a threaded post and/or a recess for receiving a tool. The device may further include a lock nut configured to mount on the threaded post and additionally secure the device to the tulip. In various embodiments, one or more surface of one or more component may include a feature, wherein the surface feature is useful for maintaining mechanical interface with a surface on a second component, and the surface feature may be selected from splines, knurls, interruptions, and protrusions.

In various aspects, a system for engaging a tulip structure is disclosed, the system including an adaptor configured to engage a tulip and including a channel configured to accept and hold a spinal rod, an adaptor set screw; and a tulip set screw. In various embodiments, the adaptor set screw and/or the tulip set screw may include a threaded post. In some embodiments, the system may further include a lock nut for securing the adaptor to the tulip, and the lock nut may be configured to mount to the threaded post. The system may further include one or more tools for engaging one or more of the screws and/or nut. The disclosed system may include one or more surfaces of one or more components with a surface feature useful for maintaining mechanical interface with a surface on a second component, and the surface feature may be selected from splines, knurls, interruptions, and protrusions.

In various aspects, a screw-type fastening device for connecting a first component and a second component is disclosed. The device including a first coupling assembly proximate a first end, and a first fastener surface, wherein the first fastener surface includes threading configured to threadably engage an internal threaded portion of a channel of the second component. In various embodiments, the first coupling assembly may be a threaded post, extending from the first end away from the fastening device, or a threaded channel, extending from the first end to the interior of the fastening device. In threaded post embodiments, the post may be configured to receive a lock nut, and in threaded channel embodiments, the channel may be configured to receive a lock screw. In many embodiments, the second component may be an adaptor for example a SideCAR adaptor and the first component may be a SideCAR-to-SideCAR adaptor.

In various aspects, an adaptor device for engaging a tulip screw is disclosed. The adaptor device includes a yoke coupled to the tulip screw, wherein a first channel capable of receiving a first spinal rod is defined between the tulip and the yoke, a second channel capable of receiving a second spinal rod extending from the yoke, and an engagement feature extending from the device and engaging the first spinal rod.

In various aspects, an adaptor coupled to a tulip screw is disclosed. The adaptor includes an engagement structure to engage a first spinal rod retained by the tulip screw, and a channel extending from the adaptor away from the tulip screw and shaped to receive a second spinal rod.

In various aspects, a device for engaging a tulip screw is disclosed. The device includes a yoke extending over a top of the tulip screw and an engagement structure to secure the device to a spinal rod, wherein the engagement structure is axially spaced from the yoke.

In various aspects, a rotatably adjustable adaptor coupled to a tulip screw is disclosed. The rotatably adjustable adaptor includes a yoke extending over a top of the tulip screw, an extension extending from a side of the yoke away from the tulip screw, the extension defining a first portion of a joint, a rotatably adjustable channel shaped to receive a spinal rod and defining a second portion of a joint, the second portion coupling to the first portion to define the joint, wherein the rotatably adjustable channel can be rotatable and angled about the joint to define a plurality of paths for the spinal rod, relative a rod in the tulip.

### BRIEF DESCRIPTION OF THE DRAWINGS

FIG. 1A depicts an exploded view of one embodiment of the disclosed devices and systems.

FIG. 1B depicts a cross sectional view of the embodiment shown in FIG. 1A.

FIG. 2A depicts one embodiment of the disclosed devices and systems engaged with a spinal rod and pedicle screw.

FIG. 2B depicts a side elevation view of the embodiment in FIG. 2A, illustrating a distance between spinal rods (‘D’) and height offsets between the rods (‘h’).

FIG. 3 depicts one embodiment of the disclosed devices and systems comprising a locking nut configured to engage a threaded post on the tulip set screw.

FIG. 4 depicts one embodiment of the disclosed devices and systems comprising a locking nut configured to engage a threaded post on the tulip set screw.

FIG. 5 depicts one embodiment of the disclosed devices and systems, in this figure the distance between spinal rods and height offsets between the rods is shown.

FIG. 6 depicts a side perspective view of one embodiment of the disclosed depicts one embodiment of the disclosed devices and systems engaged with a spinal rod and pedicle screw.

FIG. 7 depicts a perspective top view of one embodiment of the disclosed depicts one embodiment of the disclosed devices and systems engaged with a spinal rod and pedicle screw.

FIG. 8 depicts one embodiment of the disclosed tulip set screw, yoke, and lock nut.

FIG. 9 depicts one embodiment of the disclosed tulip set screw.

FIG. 10 depicts a sectional view of one embodiment of the disclosed tulip set screw, lock nut, and tool.

FIG. 11 depicts one embodiment of the disclosed tool engaging an adaptor or tulip set screw.

FIG. 12 depicts a section view of one embodiment of the disclosed tool and tulip set screw, showing tool receiving the threaded post of the set screw.

FIG. 13 depicts one embodiment of the disclosed lock nut with a hexagonal recess for accepting and engaging a similarly shaped tool.

FIG. 14 depicts one embodiment of the disclosed adaptor yoke and tulip set screw, in this embodiment a snap feature is also shown.

FIG. 15 depicts one embodiment of the disclosed adaptor and set screws with surface features for maintaining a secure interface with the adaptor.

FIG. 16 depicts one embodiment of the disclosed lock nut having surface features for maintaining a secure interface with corresponding surface.

FIG. 17 depicts one embodiment of the disclosed lock nut including a threaded post for engaging a set screw.

FIG. 18 depicts one embodiment of the disclosed adaptor or tulip set screw comprising a channel through the screw, which in this embodiment is threaded, for accepting a threaded post of another set screw or lock nut.

FIG. 19 depicts one embodiment of the disclosed adaptor or tulip set screw comprising a channel through the screw, which in this embodiment is threaded, for accepting a threaded post of another set screw or lock nut.

FIG. 20 depicts a perspective view of another embodiment of the disclosed devices and systems.

FIG. 21A depicts a cross sectional view of the embodiment of FIG. 20.

FIG. 21B depicts perspective view of a lock nut and tulip set screw of the embodiment of FIG. 20.

FIG. 22 depicts a perspective view of the tulip set screw of the embodiment of FIG. 20.

FIG. 23 depicts an elevation view of the tulip set screw of the embodiment of FIG. 20.

FIG. 24 depicts a top plan view of the tulip set screw of the embodiment of FIG. 20.

FIG. 25 depicts a perspective view of the lock nut of the embodiment of FIG. 20.

FIG. 26 depicts a top-side perspective view of the adaptor including a snap structure and a lock nut of an embodiment of the SideCAR adaptor.

FIG. 27 depicts a rear-side perspective view of the adaptor and lock nut of the embodiment of FIG. 26.

FIG. 28 depicts a cross sectional view of FIG. 26 taken along line 28-28.

FIG. 29 depicts a cross sectional view of the adaptor of FIG. 26 with a tulip set screw, lock nut, and adaptor set screw engaged with a tulip.

FIG. 30A depicts a close perspective view of the adaptor showing a filleted edge of the embodiment of FIG. 26.

FIG. 30B depicts a side perspective view of the assembled disclosed devices and systems of FIG. 26, wherein a spinal rod is positioned in the SideCAR and the tulip is shown as a semi-transparent feature, and an example applied load on the system

FIG. 30C depicts a finite element analysis showing stress concentrations on an adaptor with a filleted edge under the applied load of FIG. 30B.

FIG. 30D depicts a finite element analysis showing stress concentrations on an adaptor without a filleted edge under the applied load of FIG. 30B.

FIG. 31A depicts a side perspective view of an adaptor with a channel arranged at an offset angle.

FIG. 31B depicts a side perspective view of an adaptor with a channel at an offset angle different from that depicted in FIG. 31A.

FIG. 32 depicts a vertebral column showing embodiments of the disclosed devices and systems installed.

FIG. 33A depicts a top perspective view of an embodiment of the adaptor with the channel axially offset from the yoke.

FIG. 33B depicts a vertebral column showing embodiments of the disclosed devices and systems installed, including an embodiment of the adaptor, wherein the channel is axially offset from the yoke, similar to that seen in FIG. 33A.

FIG. 34 depicts a perspective view of an embodiment of the disclosed devices and systems, and showing rods positioned in tulip and SideCAR, wherein the disclosed SideCAR device includes outrigger structures.

FIG. 35 depicts a side view of the embodiment of FIG. 34.

FIG. 36 depicts a top view of the embodiment of FIG. 34.

FIG. 37 depicts a perspective view of an embodiment of the disclosed devices and systems, wherein the SideCAR includes structures for accepting removable outrigger structures, also shown.

FIG. 38 depicts an elevation view of the embodiment of FIG. 37.

FIG. 39 depicts a top view of the embodiment of FIG. 37.

FIG. 40 depicts a cross sectional view taken along lines 40-40 of FIG. 39.

FIG. 41 depicts a perspective view of an embodiment of the disclosed devices and systems, wherein the disclosed SideCAR devices includes outrigger structures and engagement features.

FIG. 42 depicts an elevation view of the embodiment of FIG. 41.

FIG. 43 depicts a top view of the embodiment of FIG. 41.

FIG. 44 depicts a cross sectional view taken along lines 44-44 of FIG. 41.

FIG. 45 depicts a perspective view of an embodiment of the disclosed devices and systems, wherein the disclosed SideCAR device includes engagement structures for installing and securing extension features.

FIG. 46 depicts an elevation view of the embodiment of FIG. 45.

FIG. 47 depicts a top view of the embodiment of FIG. 45.

FIG. 48 depicts a cross sectional view taken along lines 48-48 of FIG. 47.

FIG. 49 depicts a perspective view of an embodiment of the disclosed devices and systems, wherein the disclosed SideCAR device includes engagement structures for installing and securing extension features.

FIG. 50 depicts an elevation view of the embodiment of FIG. 49.

FIG. 51 depicts a top view of the embodiment of FIG. 49.

FIG. 52 depicts a perspective view of an embodiment of the disclosed devices and systems, wherein the disclosed SideCAR includes a rotatably adjustable channel for defining a range of spinal rod paths.

FIG. 53 depicts a front elevation view of the embodiment of FIG. 52.

FIG. 54 depicts a cross sectional view of the embodiment of FIG. 52 taken along line 54-54.

FIG. 55 depicts a perspective view of the embodiment of FIG. 52 with the channel rotated to a second position.

FIG. 56 depicts a front elevation view of the embodiment of FIG. 52 with the channel rotated to a second position.

FIG. 57 depicts a top view of the embodiment of FIG. 52 with the channel rotated to a second position.

### DETAILED DESCRIPTION

Disclosed herein are devices, systems, and methods for securing additional spinal rods to existing or previously installed hardware, specifically tulip structures, spinal rods, or a combination of both tulip structures and spinal rods. The disclosed devices, systems, and methods may include a (1) adaptor body; (2) adaptor set screw; (3) tulip set screw; and (4) lock nut or lock screw. Additional examples may include (5) outrigger structures, (6) engagement features or structures, and/or other features/devices that may connect the SideCAR to an existing spinal rod. Adaptors are designed to conform and securely connect to various tulip designs, accept various reduction tool configurations, and position the second rod at predetermined offsets from the existing rod and tulip. The disclosed devices and systems may be referred to as ‘SideCAR’ devices and systems.

Rods, screws, and specialized hardware (such as complex geometry mechanical nuts and rod-to-rod connectors) are used in orthopedic and spinal fixation procedures. Typically, to create complex fixation geometry, surgeons will connect to and build from a rod, using rod-to-rod connectors. Because spinal rods are generally cylindrical, rod-to-rod connectors may allow for radial movement of the second rod about the existing rod. Fracture or breakage of a rod will also compromise complex fixations based on rod-to-rod connections. The presently disclosed SideCAR devices, systems, and methods provide for secure connections to existing fixation hardware. In most embodiments, the disclosed system and devices are configured to connect to the pedicle screw, for example the tulip structure of existing pedicle screws.

The disclosed devices, systems, and methods are useful in attaching, connecting, and adapting to or atop existing tulip structures. This can be performed during an initial fixation procedure, or a revision surgery, where the broken hardware is corrected or replaced, or where geometry is adjusted. Surgeons can install the SideCAR to existing tulip structures, without removal of the existing rod secured by the tulip. Additional rods are then attached to the SideCAR device, strengthening the existing structure. Addition of SideCAR-based rods may be performed in a single, original fixation procedure, or as part of a revision procedure (where the SideCAR, new rod and other hardware are added to an existing tulip and rod construct created during an earlier spinal fixation procedure).

While devices may be described as existing or previously installed, such references may be for identification purposes only. It is understood that the recited structures and devices may be implanted or installed in a variety of orders. For example, reference to an existing structure does not necessitate that structure having been installed in a prior surgical procedure—in some embodiments, the disclosed devices and systems may be installed concurrently with an existing structure.

The SideCAR system presently disclosed, as disclosed above, may be compatible with commonly used devices to install, remove, or adjust tulip structures and rod-to-rod connections. For example, devices such as a reduction device may be able to couple to the tulips or the rods with the SideCAR installed to adjust the position of the spinal rods or tulips. In many embodiments, a reduction instrument/reducer tool may be configured to securely and reversibly adhere, attach, engage, and/or connect to a tulip to aid in insertion and positioning of a spinal rod. In some examples, a reduction instrument or reducer tool may engage directly with the SideCAR adaptor.

The present devices, systems, and methods do not require removal of the existing tulip and existing rod, greatly aiding surgeons installing/attaching secondary rods. This will benefit patients by reducing surgical time and complexity required for connecting to or affecting existing rods.

The disclosed devices, systems, and methods may be designed to attach, connect, or conform to any tulip product, spinal rod, or related hardware. Pedicle screw and tulips from various manufacturers may possess different designs, structures, and configurations. In some embodiments, the disclosed devices and systems may also include novel tulip designs. These novel tulip designs may possess specific features for adapting, accommodate, and/or conforming to compatible SideCAR structures and systems. For example, the novel tulip and adaptor design may optimize various geometric and complementary aspects (e.g. rails, slots, etc.) on the respective tulip and SideCAR structure. These complementary aspects may aid in allowing for efficient connection/attachment. In one embodiment, the complementary features may include pressure-fit, snap-fit or interlocking structures. Additional complementary structures and geometries may allow secure and/or removable attachment (in one example, for use in revision procedures) of tulip to SideCAR without necessitating additional fastening hardware, such as a locking nut.

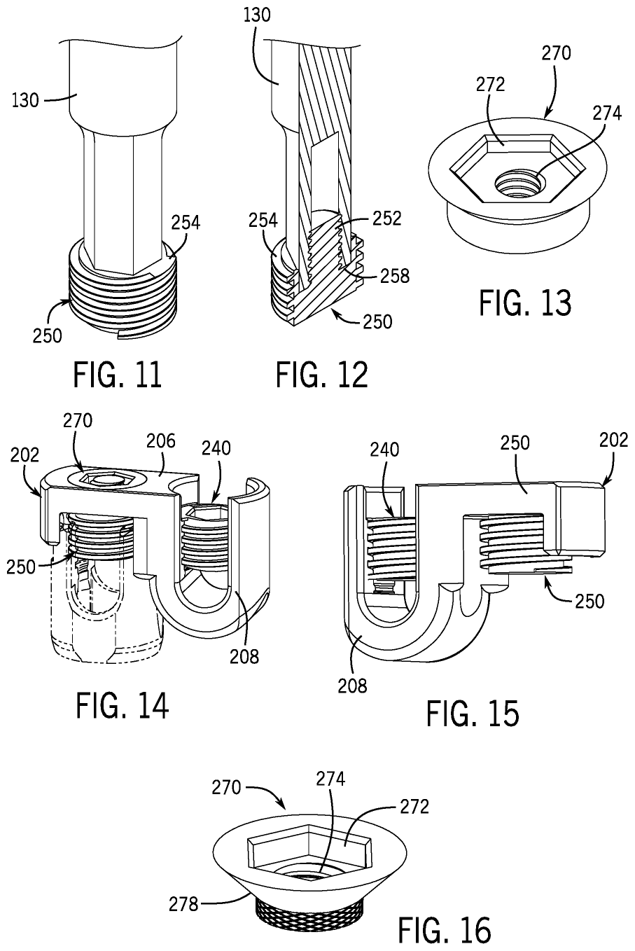

Turning to the figures, FIGS. TA-1B show one embodiment of the disclosed SideCAR device 200 and system 100. In this embodiment, the system 100 may include a SideCAR adaptor 202, a tulip pedicle screw 102, a tulip set screw 250, an adaptor set screw 240, and a lock nut 270. Inset is an embodiment of the tulip set screw 250 showing a threaded post 252 for receiving and securing the lock nut 270. Note also that the tulip set screw 250 includes a recess 256 about the threaded post 252. This recess 256 is configured to engage a tool, (e.g. the set tool 130) as may be shown in FIG. 11-12, for manipulating the tulip set screw 250.

### Adaptor—SideCAR Adaptor

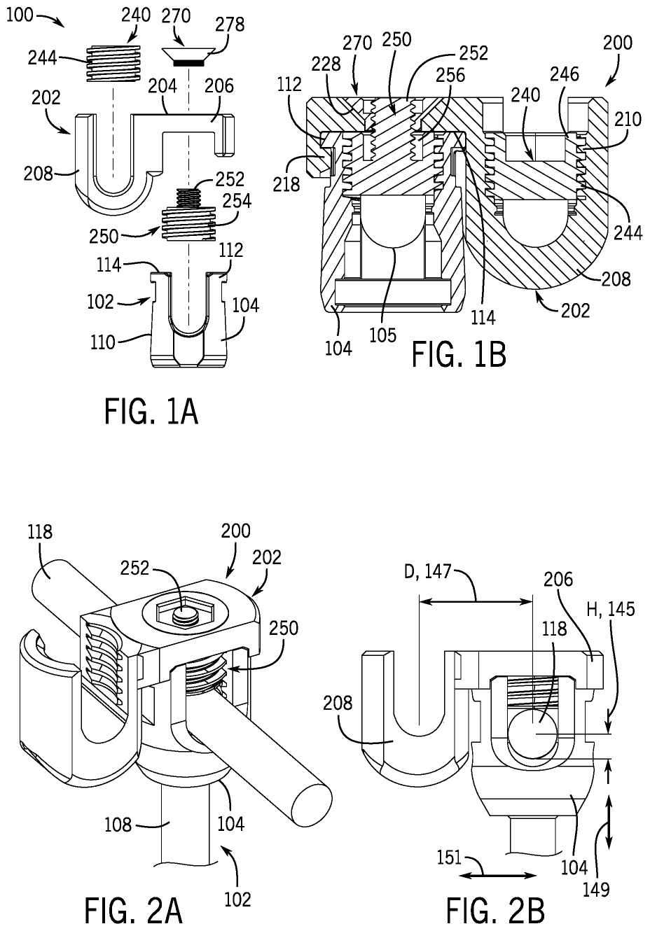

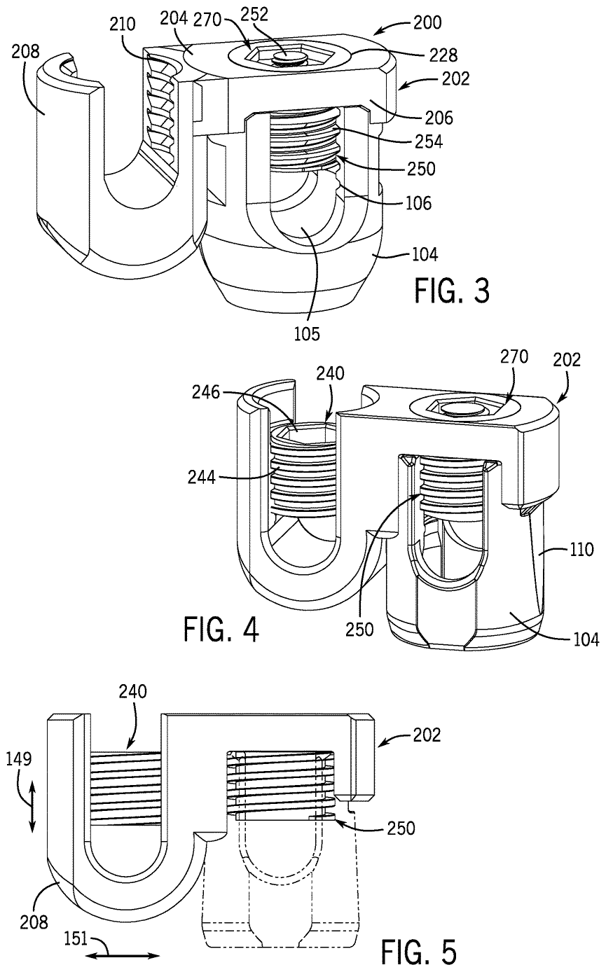

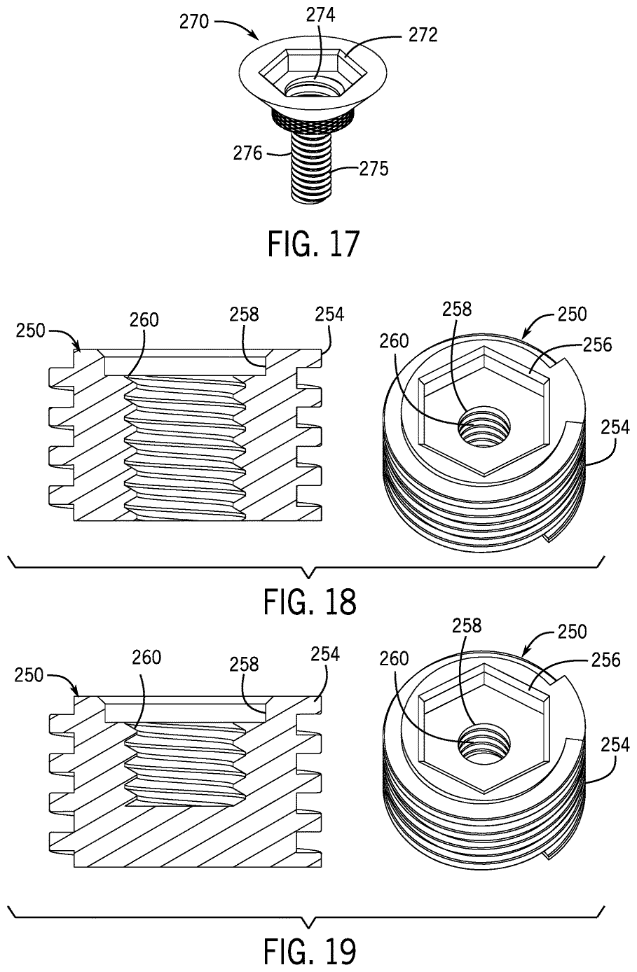

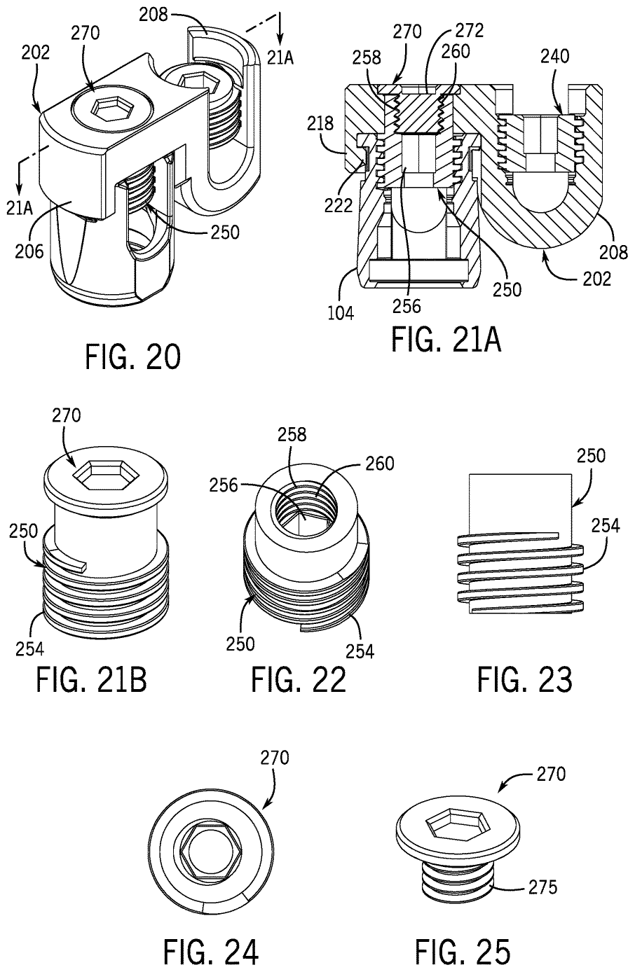

The SideCAR adaptor 202, as shown in FIGS. 1A-31, and with specific reference to FIGS. TA-1B, includes a yoke 206 for engaging the tulip 104 and a u-shaped channel or slot 208 for receiving the secondary rod 120. The channel 208 may include internal threading 210 for accepting the threads 244 of the adaptor set screw 240. In some embodiments, the yoke 206 may include a tab, lip, or snap structure 218 to engage a corresponding lip, tab, or snap structure 112 of the tulip 104. The adaptor 202 may define an aperture 228, as may be shown in FIG. 8, defined in the top exterior surface 204 of the adaptor 202 and extending through the yoke 206. The aperture 228 may be designed to allow access to the tulip 104 or to receive SideCAR hardware (for example a lock nut 270, tulip set screw 250, etc.) when the yoke 206 engages the tulip 104. The adaptor 202 may be connected to the tulip 104 solely by the yoke 206 snap fitting to the tulip 104, or otherwise connecting directly to the tulip 104, and/or by connecting to other existing hardware. In other examples, the yoke 206 may connect the adaptor 202 to the tulip 104 in combination with additional SideCAR hardware, such as a lock nut 270 or tulip set screw 250.

FIGS. 2A-2B shows a perspective view of one embodiment of the SideCAR device 200 installed on a tulip system 102 including a tulip 104 holding an existing spinal rod 118—the pedicle screw 108 is also visible in this figure, positioned below the tulip 104. As is readily apparent, the SideCAR 202 may be variously configured to hold the secondary rod 120 at various horizontal offsets 145 or distances, D, from the existing rod 118. The secondary rod 120 may be positioned at a vertical offset 145 or height, h, that is above or below the existing rod 118. The horizontal direction 151 (to modify D) and vertical direction 149 (to modify h) of configurability/adjustability are also shown with direction arrows, 151 and 149 respectively, in FIG. 5.

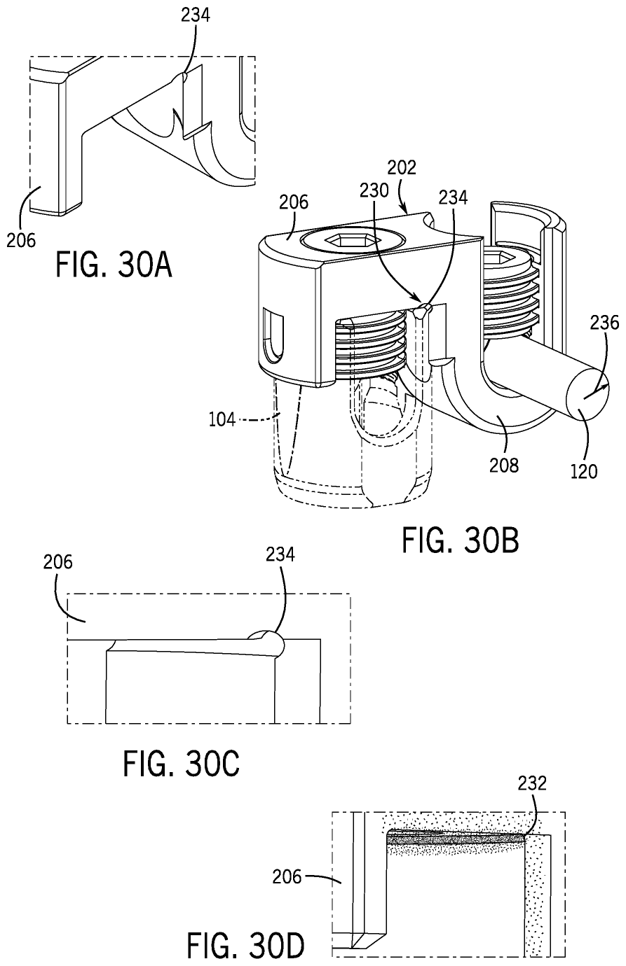

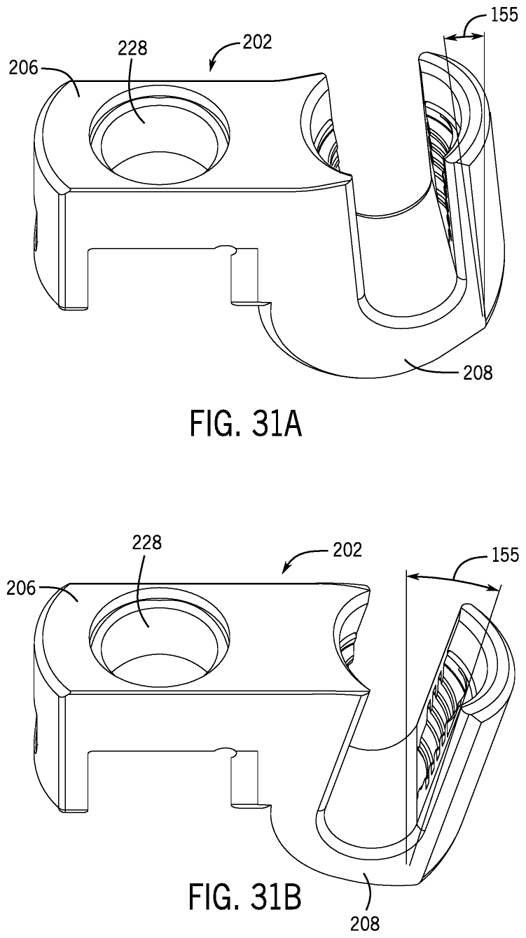

The secondary rod 120 may be placed in the U-shaped adaptor channel 208, as shown in FIG. 30B. The adaptor channel 208 may have a similar shape or profile as that of the tulip channel 105. Positioning the secondary rod 120, relative to the existing rod 118, may be accomplished by creating a family of SideCAR adaptors 202 of similar design but having varied horizontal 147, angled 155, axial, or vertical offsets 145 from the yoke 206 or existing rod 118 to the channel 208. In some examples, as shown in FIGS. 31A and 31B, the adaptor channel 208 may define an angular offset 155, such as angularly offset inward, FIG. 31A, or angularly offset outward, FIG. 31B, relative to the yoke 206. In some examples, as may be shown in FIGS. 33A and 33B, the SideCAR adaptor 202 may extend from the tulip 104 at an angle relative to tulip such that the channel 208 of the SideCAR 202 is axially offset 157 from the yoke 206 or the channel 105 of the tulip 104.

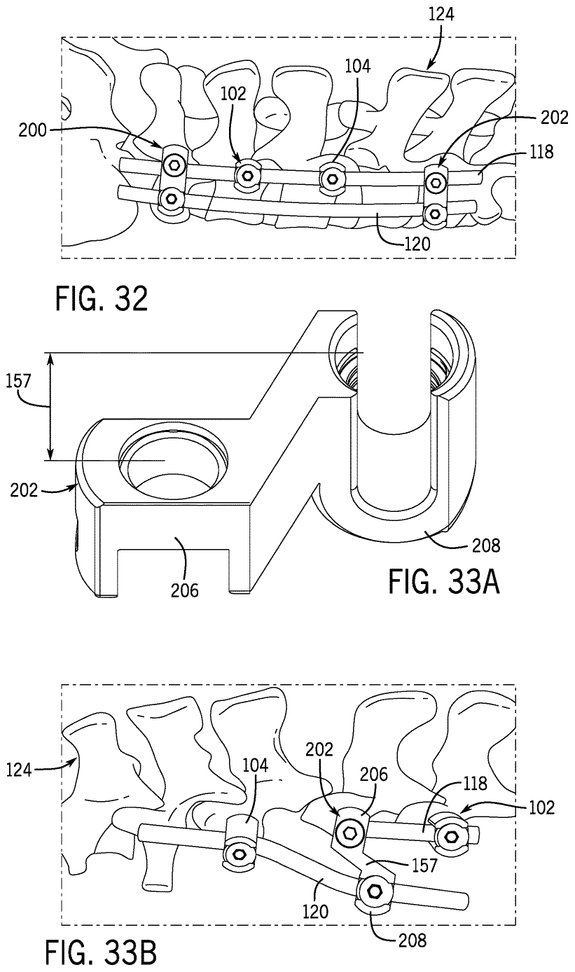

FIG. 32 depicts a vertebral column 124 showing embodiments of the disclosed devices and systems 200 installed. FIG. 33B depicts a vertebral column 124 showing embodiments of the disclosed devices and systems 200 installed, including an embodiment of the adaptor 202, wherein the channel 208 is axially offset 157 from the yoke 206, similar to that seen in FIG. 33A.

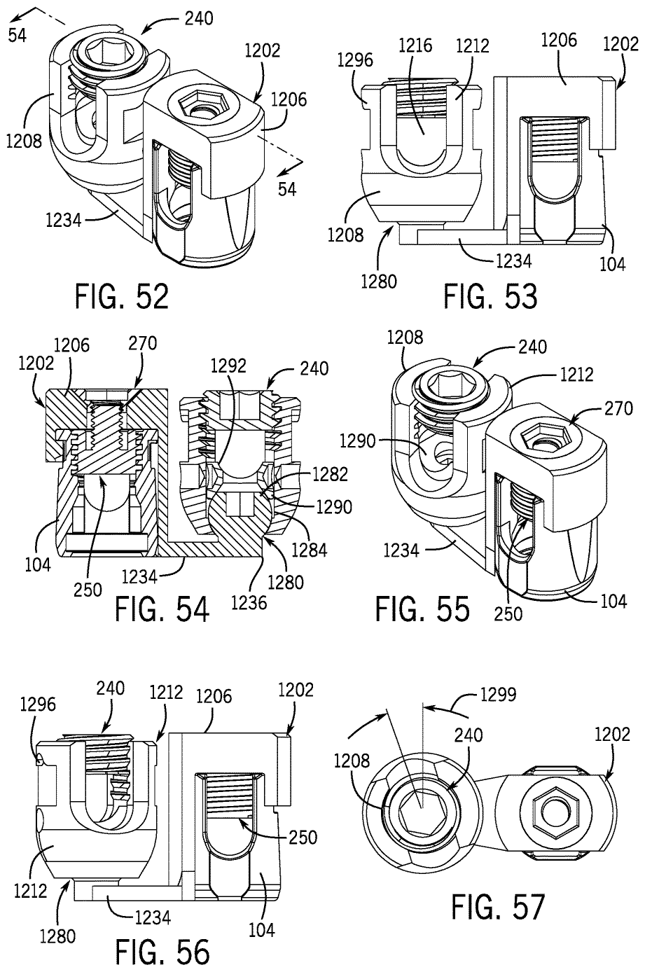

In various embodiments, an adjustable SideCAR adaptor is disclosed. In these embodiments, the surgeon may select and set the desired offsets between rods 118, 120 before and/or during a procedure. The selected offsets may be set to discrete, pre-set positions, or, in some embodiments the adaptor offset may be configured with continuous adjustability within a given range defined by the adaptor, wherein different adaptors may have different ranges for horizontal offsets (D) 147, vertical offsets (h) 145, or other offset settings. For example, as shown in FIG. 52-57, a component of the adaptor 1202, such as the channel 1208, may be rotatable relative to the adaptor 1202 to adjust a path direction of a spinal rod by an angle 1299 in the channel 1208. The variations of offsets and ranges of possible configurations of the SideCAR adaptor provide additional flexibility to the medical professional installing the SideCAR, and may assist in aligning the spinal rod systems for a variety of different patients or clinical needs.

The SideCAR adaptor 202 may secure to the tulip 104 by engaging one or more sides 110, structures 112, or positions of the tulip 104 for added support and stability. For example, the adaptor yoke 206 may include one or more snap structures 218 that may engage a corresponding snap or engagement structure 112 of the tulip 104. The snap structure 218 may be a section or portion of the adaptor yoke 206. The snap structure 218 may define a detent 222 or other feature and configured to receive or engage the tulip 104.

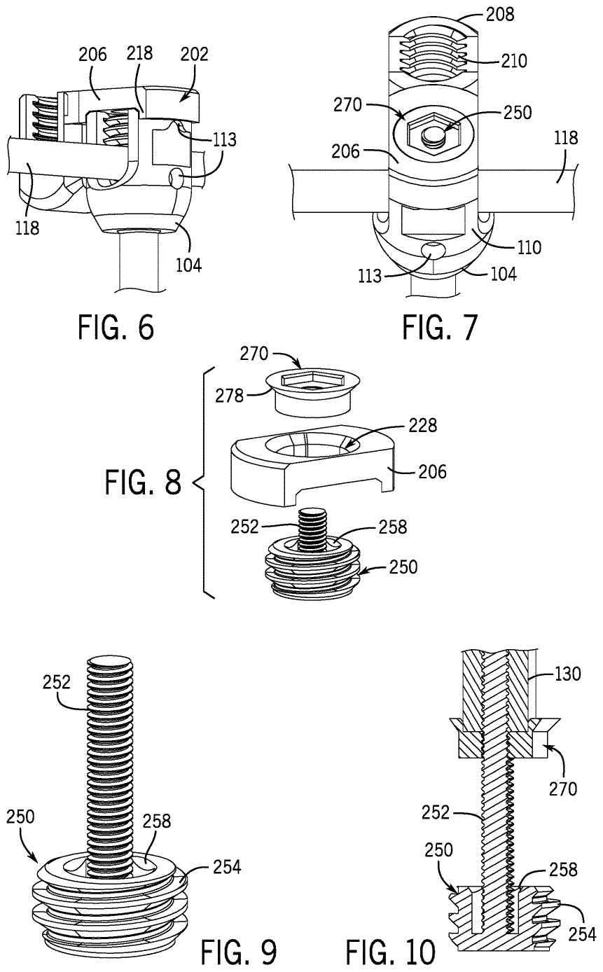

The adaptor yoke 206 may extend downward from the top 114 of the tulip 104 to various distances. In some embodiments, the tulip 104 may include one or more structures, holes, lips, recesses, etc. 112 that may be accessible for future adjustment. For example, FIGS. 6 and 7 show holes and features 113 at the side of the tulip 104 that may receive an adjustment tool, such as a reducer tool/reduction instrument, and the yoke 206 may not extend to those holes 113. In some examples, the disclosed SideCAR devices 200 may directly receive the adjustment tools, such as at the yoke 206 or adjacent the channel 208 of the adaptor 202. In other embodiments, the yoke 206 may extend to or below such a hole 113 (or other structure) to aid in securing the SideCAR adaptor 202 to the tulip 104—for one example with a snap-on structure 218, which may help in reducing movement of the SideCAR adaptor 202 relative to the tulip 104. The SideCAR adaptor 202 may be installed on every existing tulip 104, or only on a selected group of the existing tulips 104, as shown in FIG. 32 or 33B.

With reference to the snap structure 218, a snap detent 222 may define an entrance angle 224 and an exit angle 226, as shown in the example depicted in FIG. 28. Generally, an angle greater than 90 degrees relative to the yoke 206 may better facilitate engagement or disengagement, an angle of approximately or equal to 90 degrees may prohibit or limit engagement or disengagement without torqueing or distorting the SideCAR adaptor 202. Limiting disengagement may enhance retention of the SideCAR adaptor 202 to the tulip 104. An angle less than 90 degrees may provide improved retention properties. Accordingly, in some examples the entrance angle 224 of the snap detent 222 may be greater than 90 degrees while the exit angle 226 may be approximately 90 degrees or less relative to the side of the yoke 206.

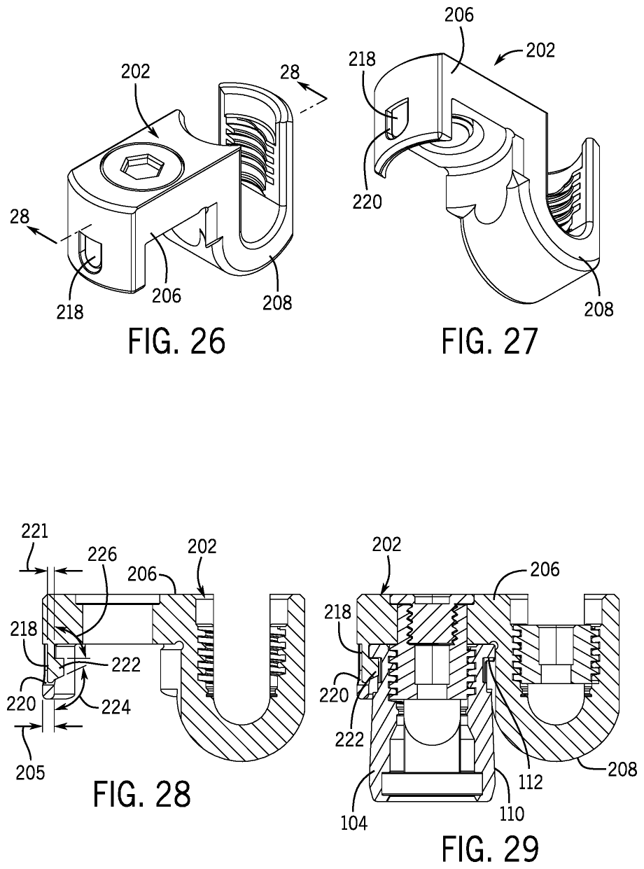

In some examples, as may be shown in FIGS. 26-29, an aperture or cut 220 may be defined by the adaptor 202 around the yoke snap structure 218. In one example, the cut or aperture 220 may be U-shaped and pointed towards the tulip 104 when viewed from a side. The thickness of the snap structure 218 of the adaptor 202 around the snap detent 222 may also be varied. As shown in FIG. 28, the yoke snap structure 218 may have a thickness 221 less than the thickness 205 of the surrounding SideCAR adaptor 202. The cut 220 around the yoke snap structure 218 and/or varying the thickness 221 of the yoke snap structure 218 may increase the flexibility or allowable deformation of the yoke snap structure 218. Increasing the flexibility or allowable deformation may result in an easier installation or removal of the SideCAR adaptor 202 on a tulip 104.

The SideCAR adaptor 202 may define or include multiple edges and locations 230 where portions of the SideCAR adaptor 202 intersect. These intersections 230 may experience stress concentrations after installation or during movement. In one example, a load 236 may be applied at the secondary rod 120 creating stress concentrations, with greater stress, indicated by a darker shading, around the sharp edge 232 as shown in FIG. 30D, with a direction of the load indicated by the arrow 236 in FIG. 30B. To reduce the stress concentrations, the intersections 230 may include or define fillet edges 234, or other stress-reducing features. Where the stress reducing feature is a fillet edge 234, the intersecting surfaces define a rounded edge rather than sharp angled corners 232, as shown in FIGS. 26-30C. The filleted edge 234 may result in reduced stress concentrations at the edge 230 and a reduced risk of the adaptor 202 being damaged during or after installation.

SideCAR-to-SideCAR adaptors are also disclosed. In these embodiments, a SideCAR-to-SideCAR adaptor connects to a previously installed SideCAR adaptor to extend the possible connections. In these embodiments, similar devices and features, for example those that aid in attaching/connecting/conforming between a tulip and a SideCAR adaptor 202 are useful with the disclosed SideCAR-to-SideCAR adaptor. For example, the adaptor set screws 240 and lock nuts 270, as described above for tulip 104 to SideCAR adaptor 202 connections, may be useful in securing a SideCAR-to-SideCAR adaptor to a SideCAR adaptor 202. These embodiments further increase the range of options for the spinal surgeon to construct fixation hardware configurations during original or revision surgeries.

### Adaptor Set Screw

The disclosed devices and systems may include an adaptor set screw 240 for insertion into the adaptor channel 208 securing the secondary rod 120 positioned therein, as may be shown in at least FIG. 4. The adaptor set screw 240 includes external threading 244 corresponding to and designed to engage with the internal threading 210 of adaptor channel 208. In various embodiments, the adaptor set screw 240 may include a structure 246 for engaging a set tool 130 that may be useful in screwing and securing the adaptor set screw 240 into the adaptor channel 208 and contacting the secondary rod 120. In various embodiments, the adaptor set screw 240 may further include a threaded post, wherein the threaded post may be configured to accept a lock nut, which may be the same as or similar to lock nut 270.

As noted above, various embodiments of the devices and systems may include a second device, a SideCAR-to-SideCAR adaptor. In these embodiments, the adaptor set screw 240 may include one or more features for engaging and securing the SideCAR-to-SideCAR adaptor to the SideCAR adaptor 202, for example a channel or threaded post for engaging a corresponding feature on a second lock nut.

### Tulip Set Screw

The disclosed devices and systems may include a tulip set screw 250 for inserting into the tulip 104 and securing the existing rod 118. Various examples of the tulip set screw 250 may be shown in at least FIGS. 8-12 and 18-19. The tulip set screw 250 includes threads 254 that correspond to and engage with the internal threading 106 of the tulip 104. The tulip set screw 250 may further engage with or be received within an aperture 228 of the SideCAR adaptor 202.

In various embodiments, the tulip set screw 250 may include a recess 256 for accepting a set tool 130 that may be useful in screwing and securing the tulip set screw 250 into the tulip channel 208 and contacting the existing rod 118. The tulip set screw 250 may further include a threaded post 252, wherein the threaded post 252 may be configured to accept a lock nut 270. The tulip set screw 250, in some embodiments, may include a channel 258 for engaging a threaded post of a lock nut 270. For example, the channel 258 of the tulip set screw 250 may define threading 260. In these embodiments, the channel 258 may be through the set screw 250, or may terminate within the body of the set screw 250. Further, the recess 256 for accepting a set tool 130 and the channel 258 for engaging the lock nut 270 may be defined by the same aperture. For example, as shown in FIG. 21, the lower portion of the aperture may define the recess 256 and the upper portion of the aperture may define threading 254 to receive a feature of the lock nut 270.

### Lock Nut

The disclosed devices and systems may include a lock nut 270. Various examples of the lock nut 270 may be shown in the figures. The lock nut 270 may be designed to engage the tulip set screw 250 and tighten the SideCAR with tulip. In some embodiments, the lock nut 270 may include a central channel 272 with internal threading 274 configured to correspond to and engage threading 254 on a threaded post 252 of the tulip set screw 250. In other examples, the lock nut 270 may define external threading 276 and may be received in a channel 258 of the tulip set screw 250. In further examples, the lock nut 270 may be received within the aperture 228 of the SideCAR adaptor 202 and extend through the aperture 228 to engage with the tulip set screw 250. The lock nut 270 may also engage threading of the SideCAR aperture 228.

The lock nut 270 may include an external chamfer 278 for engaging a surface on another device, for example a corresponding chamfer on the SideCAR adaptor. The chamfer 278 may aid in tightening the nut 270 and/or the SideCAR adaptor 250 to the tulip 104. In some embodiments, the lock nut 270 may include a threaded post 275 for engaging a set screw 250 having a corresponding channel 258.

### Set Tool

The disclosed devices and system may include one or more tools 130 to aid in installing the tulip set screw 250, adaptor set screw 240, and lock nut 270. As depicted in FIGS. 10-12, the disclosed tool 130 may include a center channel 132 for receiving a threaded post on the tulip set screw 250. The tool 130 may have various outer configurations—for example hexagonal, cam, Torx, star, etc. and the adaptor set screw 240, tulip set screw 250 and lock nut 270 may include corresponding recesses for receiving and engaging the tool 130. In various embodiments, separate tools 130 may be designed for engaging the adaptor set screw 240, tulip set screw 250, and lock nut 270. In most embodiments, the disclosed tools 130 are sized to fit in a reduction device, for example novel reduction devices or a reduction device configured to correspond to the existing tulip 104.

The disclosed tool 130 may include one or more additional structures. In some embodiments, the tool 130 can include or be configured to include a handle, torque driver, and/or torque wrench for tightening a screw or nut to a specified torque value.

As disclosed above, the SideCAR devices and systems 100 may include one or more snap-fit, pressure-fit, or like structures/features. The snap-fit structures/features may be configured to work together with the screw and the fit of the SideCAR 202 to the tulip 104. In most embodiment, the SideCAR adaptor 202 is configured with a yoke 206 designed to conform to a specific tulip 104. This may help ensure a snug and secure fit between the tulip 104 and SideCAR adaptor 202. These features may be also configured to work together with the set screws 240 or 250 and fit the SideCAR 202 to other SideCAR hardware.

Any of the features, components, and/or parts, including the arrangements and configurations thereof shown in any one of FIGS. 1A-33 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 1A-33.

SideCAR with Rod Engagement Features and Structures

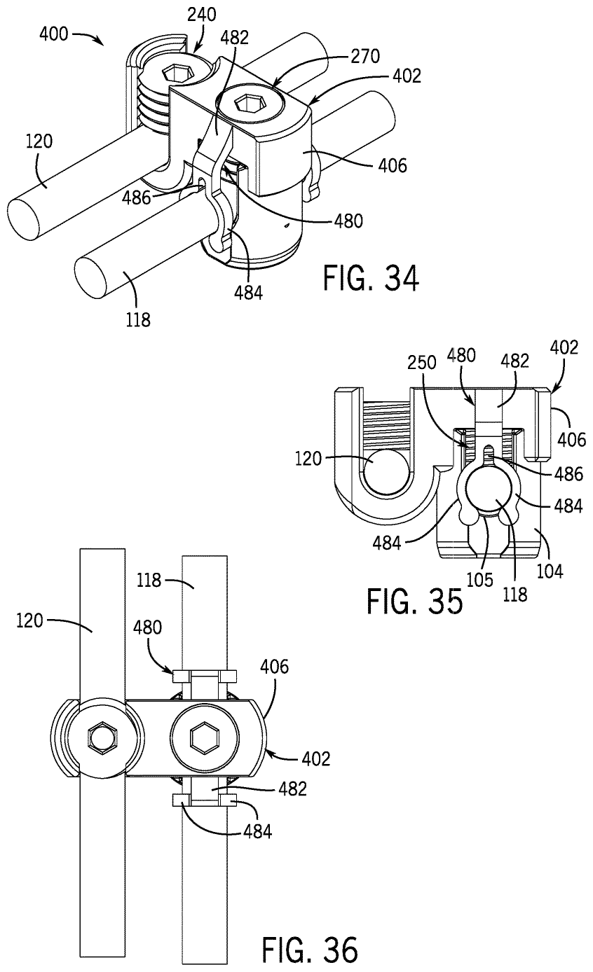

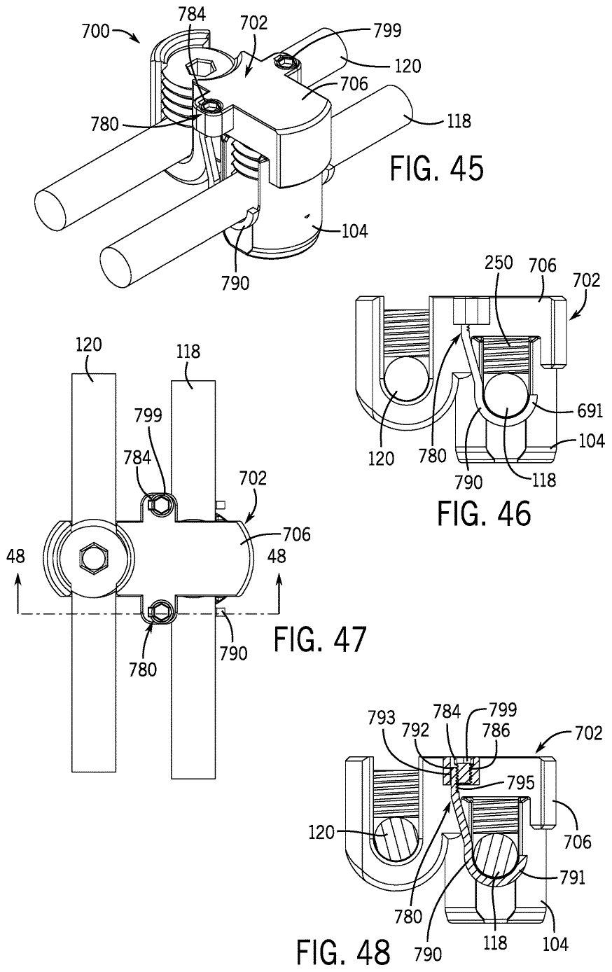

With reference to FIGS. 34-51, additional examples of the SideCAR adaptor may include engagement features or structures to aid in contacting and/or engaging a spinal rod 120 positioned in the tulip channel 105. In some embodiments, these engagement features and structures may include outrigger structures. In many embodiments, these engagement features may securely and reversibly engage, contact, and/or grasp the existing spinal rod 118. The features may aid in coupling the SideCAR to the existing spinal rod 118, as well as the tulip 104, and may help improve the securement of the SideCAR adaptor to the existing hardware. The disclosed rod engagement features may also assist in adjusting or orienting one or more of the existing spinal rod 118, the SideCAR, and the secondary spinal rod 120.

The SideCAR adaptors described herein may include all or some of the previously described rod engagement features. For example, as shown in FIG. 34, the additional examples may include a tulip screw 102, an existing spinal rod 118, a secondary spinal rod 120, adaptor set screws 240, tulip set screws 250, lock nuts or screws 270, and/or a SideCAR adaptor. In other examples, the SideCAR may include only an adaptor set screw 240 and attach to the existing hardware solely by snapping to either or both of the tulip 104 or the existing spinal rod 118.

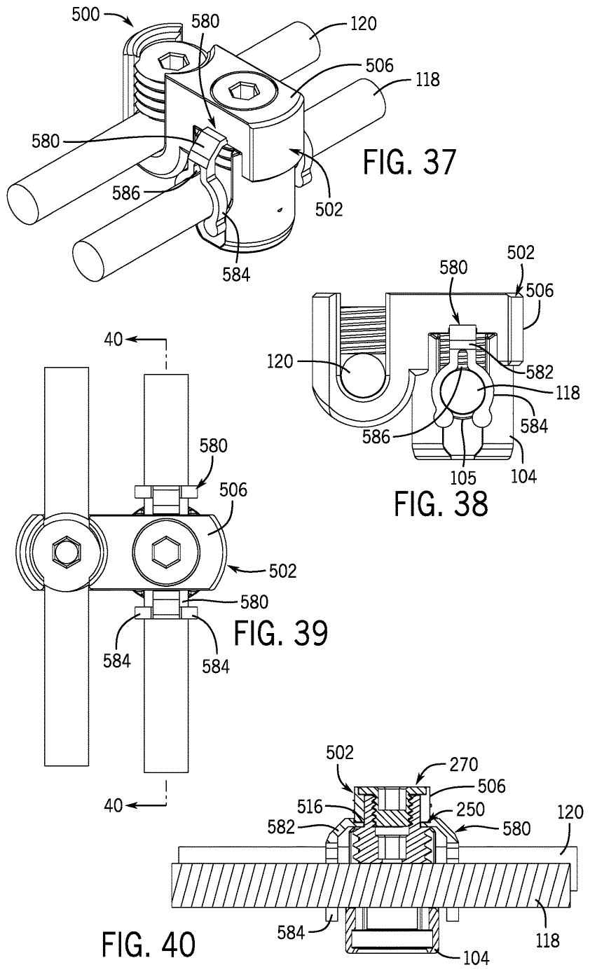

In one example, as shown in FIGS. 34-40, the SideCAR adaptor system 400 may include a sideCAR adaptor 402 and rod engagement features that may include one or more outrigger structures 480. In some of these embodiments, the outrigger structures 480 may be integrated or formed with the SideCAR adaptor 402, as shown in FIGS. 34-36. In other examples of the SideCAR adaptor system 500 the outrigger structures 580 may be removably attached, as shown in FIGS. 37-40. In many embodiments, the outrigger structures 480 may extend from the SideCAR adaptor 402 to contact/engage the existing spinal rod 118. In some embodiments, the outrigger structure 480 may removably clip or snap to the existing spinal rod 118, for example, when the SideCAR adaptor 402 is coupled to the tulip 104.

The outrigger structure 480 may include an arm-like structure 482 extending downward toward the pedicle screw or tulip 104 or tulip channel 105. The outrigger structures 480 may also extend axially outward from the adaptor yoke 406. The downward extending portion 482 of the outrigger structure may bifurcate to define two or more portions 484 separated by a gap 486. The existing spinal rod 118, positioned within the tulip channel 105 may be received within gap 486 and the portions 484 may be configured to flex around, retain, and secure the spinal rod 118. The SideCAR adaptor 402 may also be secured to the tulip 104 in any of the previously described ways.

The SideCAR adaptor 402 and outriggers 480 may be configured to maintain the position of the existing spinal rods 118 and/or prevent or resist movement of the existing spinal rod 118 relative to the new spinal rod 120 positioned within the SideCAR channel 408. In other examples, the outriggers 480 may be of a certain offset to raise or move the existing spinal rod 118 relative to the tulip 104 to adjust the position of the existing spinal rod 118, and combinations thereof. In some examples, the outriggers 480 may further extend at an angle relative to the tulip 104 to match a curve within the length of the existing spinal rod 118 and/or to effectuate a curve on the new or existing spinal rod 120.

As noted above, the outrigger structures 480 may be formed integrally with the SideCAR adaptor 402, as may be shown in FIGS. 34-36. In some examples, as shown in FIGS. 37-40, the outriggers 580 may be a separate removable component. The removable outrigger 580 may similarly define an arm portion 582. The arm portion 582 may similarly bifurcate into two or more portions 584. Similarly to the SideCAR adaptor 402, the existing spinal rod 118, positioned within the tulip channel 105, may be received within gap 586 and the portions 584 may be configured to flex around, retain, and secure the spinal rod 118. The SideCAR adaptor 502 may define a recess 516 within the yoke 506 for receiving and securing the removable outrigger structure 580, as shown in FIG. 40. The tulip set screw 250 and/or lock nut 270 may be useful in securing a removable outrigger 580 to the SideCAR adaptor 502 and in turn the SideCAR system 500 to the existing spinal rod 118.

Any of the features, components, and/or parts, including the arrangements and configurations thereof shown in any one of FIGS. 34-40 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIG. 34-40.

In some embodiments, the rod engagement structures may extend outward from the side of the adaptor yoke, but not grasp the existing rod. In some embodiments, the rod engagement structures may not contact the existing rod. In these embodiments, a further, or second, engagement device/structure may secure the existing rod and rod engagement structure.

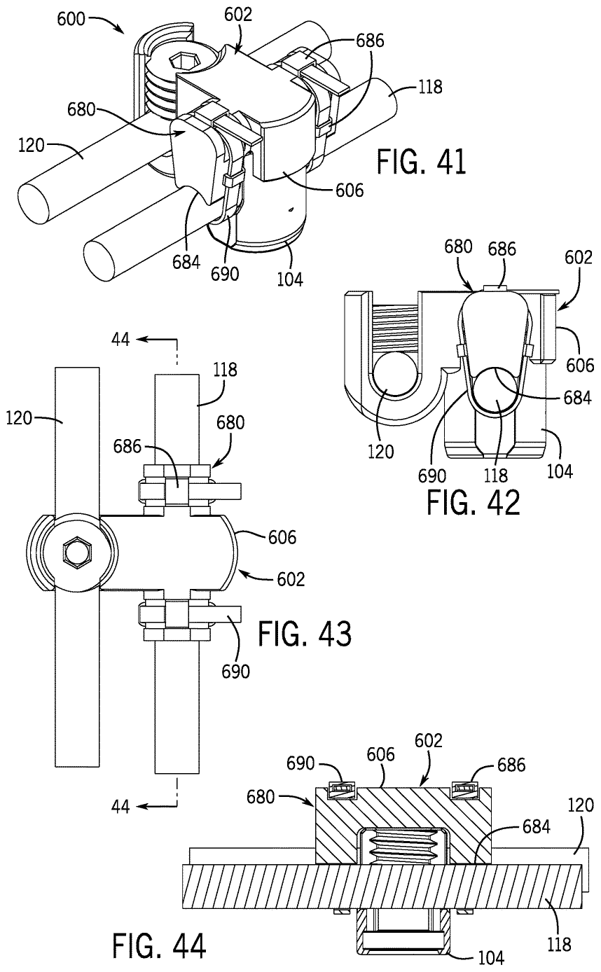

As shown in FIGS. 41-44, some examples of the SideCAR system 600 may include a first engagement structure 680 similar to the outrigger structures described above, while an additional, or second, engagement feature 690 that may secure the SideCAR adaptor 602 to the existing spinal rod 118. In some embodiments, the first rod engagement structures 680 may extend outward from the side of the adaptor yoke 606, but not grasp the existing rod 118. In some embodiments, the rod engagement structures 680 may not contact the existing rod 118. In these embodiments, the second, engagement device/structure 690 may secure the existing rod 118 and rod engagement structure 680.

The first engagement structures 680 may extend axially from the yoke 606 and parallel to the existing rod 118. The first engagement structures 680 may be spaced apart from or contact the sides of the tulip 104. In most embodiments, the first engagement structures 680 may define indents, channels, and/or other surfaces 684 shaped to conform to and/or interface with the existing spinal rod 118. The first engagement structures 680 may further define receptacles 686, such as grooves or apertures, to receive and/or secure the second engagement structures 690. In some embodiments, the additional engagement features 690, as shown in FIGS. 41-44, may then couple and secure the first engagement structure 680 to the existing spinal rod 118.

The second engagement structure 690 may surround one or both of the existing spinal rod 118 and the first engagement structure 680. In other examples, the second engagement structure 690 may only partially surround one or both of the first engagement feature 680 or existing spinal rod 118. For example, the second engagement structure 690 may be a band, cable, tie, or similar structure, and may couple the outriggers 680 or the SideCAR adaptor 602 to the existing spinal rod 118. In some embodiments, the second engagement structure 690 could be a clip or retaining ring with features to engage the rod 118 and features to engage the SideCAR adaptor 602.

Any of the features, components, and/or parts, including the arrangements and configurations thereof shown in any one of FIGS. 41-44 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 41-44.

In some embodiments, as shown in FIGS. 45-51, the rod engagement structures may be components separate from and connected to the SideCAR adaptor. These rod engagement structures may connect to or engage, but not grasp the existing spinal rod 118. The SideCAR adaptor may include or define additional features to receive or engage with the engagement structures. For example, the additional features of the SideCAR adaptor may include secondary apertures, fasteners, slots, or similar structures.

Turning to FIGS. 45-48, the SideCAR system 700 may include an engagement structure or assembly 780. The engagement structures 780 may include a hook 790. The hook 790 may be received in secondary apertures 784 defined by the SideCAR adaptor 702. The hook 790 may be included to additionally, or alternatively, secure the SideCAR adaptor 702 to the existing spinal rod 118. The hook 790 may be a structure that extends under the existing spinal rod 118 at one end 791 and attaches to the SideCAR adaptor 702 at an opposite end 792. In one example, the hook 790 may be a J-shaped hook.

In one example, the SideCAR adaptor 702 may define axially extending features 782 that define the secondary apertures 784. The secondary apertures 784 may be horizontally offset from the existing spinal rod 118, for example in the horizontal direction 151.

A fastening portion 792 of the engagement structure 790 may be inserted into a secondary aperture 784. The fastening portion 792 of the engagement structure 790 may, in one example, be smooth on a first side 793 and define threading or other coupling features 795 on a second side. For example, the hook 790 may contact or be inserted into the secondary aperture 784 with the smooth side 793 against the smooth portion 788 and include a series of thread or threading 795 on an opposite side configured to face the center of the secondary aperture. The secondary aperture 784 may further define corresponding threading 786 with the threading 795 of the fastening portion 792 of the engagement structure 790. A fastener 799 may be inserted into the secondary aperture 784 to engage one of or both of the SideCAR secondary aperture 784 and the hook 790. The fastener 799 may secure the hook 790 to the SideCAR adaptor 702. In other examples, the fastener 799 may be a nut or bolt that engages only the engagement structure 790, while the secondary apertures 784 of the SideCAR adaptor 702 may be counter sunk such that the fastener 799 may not pass through a secondary aperture 784. The fastener 799 may be rotated to pull the hook 790 through the aperture 784 and secure the engagement assembly 780 to the SideCAR 702.

The engagement assembly 780 may be positioned to exert a lifting force on the existing spinal rod 118. The lifting force may assist in adjust the existing spinal rod 118. The engagement assembly 780 may also exert a downward force on the SideCAR adaptor 702 to secure the SideCAR adaptor 702 to the existing hardware. The position of the engagement assembly 780 may be adjusted by raising or lowering the hook 790 within the secondary aperture 784. By adjusting the position of the hook 790 and/or the tulip set screw 250, the position of the existing spinal rod 118 relative to the tulip 104 may also be adjusted. The engagement assembly 780 and/or the tulip set screw 250 may allow for fine adjustments of the positions of the existing spinal rod 118. The SideCAR adaptor 702, in this example, may or may not engage with the tulip set screw 102. In one example, the SideCAR system 700 may not include a lock nut 270 or similar feature.

Any of the features, components, and/or parts, including the arrangements and configurations thereof shown in any one of FIGS. 45-48 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 45-48.

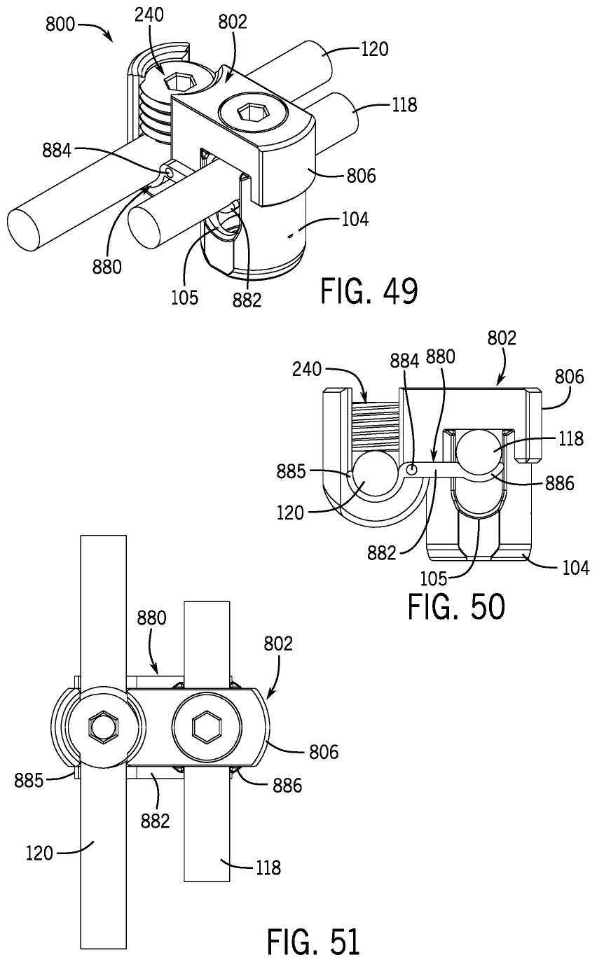

In some embodiments, an engagement structure 880 may be movable relative to the SideCAR adaptor 802. The engagement structures 880 may be connected or otherwise engaged with the SideCAR adaptor 802 and movable. For example, as shown in FIGS. 49-51, SideCAR adaptor 802 may include or connect with rotatable engagement structures 880. The engagement structures 880 may engage with both or one of the spinal rods 118, 120. When the engagement structure 880 receives or engages both spinal rods 118, 120, the engagement structure 880 may create a positional relationship between the spinal rods. The shape or length of the engagement structure 880 may define the positional relationship between the spinal rods 118, 120. The positional relationship may define a constant spacing or arrangement between the rods 118, 120. The engagement structure 880 may position one or both rods 118, 120 above the tulip channel 105 or the adaptor channel 208. The engagement structure 880 may further adjust the position of the spinal rods 118, 120 relative to the adaptor 802.

In one example, the engagement structures 880 may include a cantilever beam 882. The cantilever beam 882 may assist in securing the SideCAR adaptor 802 to the existing hardware or to assist in adjusting the position of the spinal rods 118, 120. The cantilever beam 882 may be shown in in FIGS. 49-51. The cantilever beam 882 may rotate about a connection point 884 with the SideCAR adaptor 802. The connection point 884 may be a pin or axle extending through a portion of the SideCAR adaptor 802. The connection point 884 may alternatively be a feature integral with or defined by the SideCAR adaptor 802. The pin or axle 884 may act as a fulcrum for the cantilever beam 882. The cantilever beam 882 may be shaped to receive the secondary spinal rod 120 and the existing spinal rod 118. For example, the engagement structure 880 may be curved at either end 885, 886 to match the shape of or to receive the secondary spinal rod 120 or the existing spinal rod 118. The engagement structures 880 may extend under both the existing 118 and secondary spinal rod 120. In other examples, the engagement structures 880 may extend above both or on opposite side of the spinal rods 118, 120.

When the secondary spinal rod 120 is inserted into the SideCAR channel 808, an adaptor set screw 240 may seat the secondary spinal rod 120 into the SideCAR channel 808. As the adaptor set screw 240 is rotatably moved downward, the adaptor set screw 840 may adjust the secondary spinal rod 120 downward. The engagement structure 880 may rotate, resulting in the end 886 of the cantilever beam 882 near the existing spinal rod 118 moving upward. The engagement structure 880 may grip the existing spinal rod 118 to secure the SideCAR adaptor 802 to the existing spinal rod 118 or to define the positional relationship between the spinal rods 118, 120. Further tightening of the adaptor set screw 840 may increase the pressure of the engagement structure 880 against the existing spinal rod 120 to provide securement. In other examples, as shown in FIGS. 50, the tulip set screw 250 may not be included and the tightening of the adaptor set screw 240 may move the existing spinal rod 118 upward. In other examples, adjusting the position of the tulip set screws 250 and adaptor set screws 240 may allow for fine adjustment of the positions of the spinal rods 118, 120 in relation to each other.

Any of the features, components, and/or parts, including the arrangements and configurations thereof shown in any one of FIGS. 49-51 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 49-51.

SideCAR with Rotatably Adjustable Channel

With reference to FIGS. 52-57, some embodiments of the SideCAR system 1000 may include a rotatably adjustable channel 1208. The rotatably adjustable channel 1208 may allow an adaptor 1202 to adopt various positions and thereby define a plurality of angled paths for a second, or new, spinal rod 120.

The adaptor 1202 of the present example may include a yoke 1206, an extension 1234, a joint 1280, a rotatably adjustable channel 1208, a collar 1290, and an adaptor set screw 240. The joint 1280 may provide for continuous adjustment of the rotatably adjustable channel 1208 across a range of positions and angles. In other embodiments, the joint 1280 may define a subset of predetermined positions and angles 1299. In most embodiments, the medical professional installing the SideCAR adaptor 1202 may adjust the rotatably adjustable channel 1208 around the joint 1280 to achieve a desired position or angle 1299, and may, thereafter, fix the channel position or angle 1299.

In one example, as shown in FIGS. 53, the yoke 1206 may connect to an existing tulip 104 and a portion of the yoke 1206 may extend down a side of the tulip 104. The extension 1234 may extend radially outward from the yoke 1206. The extension 1234 may define a portion of the joint 1280 at an end 1236 of the extension 1234 positioned away from the yoke 1206. In one example, as shown in FIGS. 54, the joint 1280 may be a ball and socket joint with the ball portion 1282 of the joint 1280 defined by the extension 1234 and the socket 1284 defined by the rotatably adjustable channel 1208.

The rotatably adjustable channel 1208 may define channel walls 1212 forming a U-shaped opening 1216 to receive a spinal rod, such as a secondary spinal rod 120 described above. The rotatably adjustable channel 1206 may further define an aperture 1284 at the bottom 1214 of the U-shaped opening 1216 and extending through the channel 1206. The aperture 1284 may define the socket and receive the ball 1282 of the extension 1234, defining the joint 1280.

A collar 1290 may be inserted into the U-shaped opening 1216. The collar 1290 may have an outer width or diameter sufficient to mate with the inner surfaces or walls 1212 of the channel 1208. The collar 1290 may further be a solid piece or define an opening with an inner diameter or width less than a diameter of the ball portion 1282 of the joint 1280. The collar 1290 may act to set a position of the rotatably adjustable channel 1206 relative to the yoke 1206 and prevent the ball portion 1282 of the joint 1280 from rising relative to the channel 1208. The collar 1290 may rotate with, or separate from, the channel 1208, as shown in FIGS. 55-57. The upper surface 1292 of the collar 1290 may define a shape matching the profile of the U-shaped channel 1208. A spinal rod 120 may contact the upper surface 1292 of the collar 1290 when the spinal rod 120 is inserted into the U-shaped channel 1208.

An adaptor set screw 240 may be inserted into the U-shaped channel 1208 and secure the spinal rod 120 within the channel 1208. The channel walls 1212 may further define features 1296, such as on the exterior, to receive a tool, such as a reducer tool, directly at the adaptor 1202. The tools may adjust the position of the rotatably adjustable channel 1208 to define an angled path, as may be shown in FIG. 57. The plurality of angled paths for a spinal rod 120 defined by the rotatably adjustable channel may allow the SideCAR adaptor 1202 to account for a variety of positions of tulip screws 102 in a patient or a variety of anatomical differences between patients.

Any of the features, components, and/or parts, including the arrangements and configurations thereof shown in any one of FIGS. 52-57 can be included, either alone or in any combination, in any of the other examples of devices, features, components, and parts shown in the other figures described herein. Likewise, any of the features, components, and/or parts, including the arrangements and configurations thereof shown and described with reference to the other figures can be included, either alone or in any combination, in the example of the devices, features, components, and parts shown in FIGS. 52-57.

In various embodiments, corresponding surfaces, especially surfaces that interface a moving structure/device (e.g. a screws or nut) and a stationary device/structure (e.g. the tulip 104 or SideCAR 202) may have one or more surface patterns or structures for maintaining positive and secure engagement of the corresponding surfaces. In some embodiments, the surfaces may include splines or knurling that are useful in creating more positive mechanical engagement upon installation. In some configurations the surface features can be tuned to provide different resistive forces, for example to require less torque to install and more torque to remove. In some embodiments, the surface features may be present on any interfacing surface of the tulip or SideCAR adaptor structure 100, and may include sub-structures, for example a gentle ramp in a direction of installation and a sharper ramp in direction of removal.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

1-8. (canceled)

9. A system for engaging a tulip structure, comprising:
an adaptor configured to engage a tulip and comprising:
a channel configured to accept and hold a spinal rod; and
a yoke configured to engage the tulip;
an adaptor set screw for securing a first spinal rod in the channel, wherein the adaptor set screw includes a recess for receiving a tool, and threading corresponding to threading within the channel; and
a tulip set screw for securing a second spinal rod in the tulip, wherein the tulip set screw includes a threaded post.

10. The system of claim 9, wherein the adaptor set screw comprises a threaded post.

11. The system of claim 9, comprising a lock nut for securing the adaptor to the tulip.

12. The system of claim 11, wherein the lock nut is configured to mount to the threaded post of the tulip set screw.

13. The system of claim 9, comprising a tool for engaging one or more of the adaptor set screw or the tulip set screw, wherein the tulip set screw comprises a recess for receiving a tool.

14. The system of claim 9, wherein the yoke includes an engagement feature extending from the device and engaging the second spinal rod.

15. The system of claim 14, wherein at least one surface of one component includes a surface feature, wherein the surface feature is useful for maintaining mechanical interface with a surface on a second component and the surface feature is selected from splines, knurls, interruptions, and protrusions.

16-32. (canceled)

33. A rotatably adjustable adaptor for engaging a tulip screw, the rotatably adjustable adaptor comprising:
a yoke for engaging the tulip screw,
an extension extending from a side of the yoke away from the tulip screw, the extension defining a first portion of a joint;
a rotatably adjustable channel shaped to receive a first spinal rod and defining a second portion of the joint, the second portion of the joint coupling to the first portion of the joint to define the joint;
wherein the rotatably adjustable channel can be rotatable and angled about the joint to define a plurality of paths for the first spinal rod, relative to a second spinal rod positioned within the tulip.

34. The rotatably adjustable adaptor for engaging a tulip screw of claim 33, wherein the rotatably adjustable channel defines a U-shaped opening to receive the first spinal rod.

35. The rotatably adjustable adaptor for engaging a tulip screw of claim 33, the rotatably adjustable channel is continuously rotatable about the joint.

36. The rotatably adjustable adaptor for engaging a tulip screw of claim 33, wherein the first portion of the joint is a ball and the second portion of the joint is a socket and the joint allows adjustment of the rotatably adjustable channel across a range of positions as well as angles.

37. The rotatably adjustable adaptor for engaging a tulip screw of claim 33, comprising a collar positioned within the rotatably adjustable channel for setting a position of the rotatably adjustable channel relative to the yoke.

38. The rotatably adjustable adaptor for engaging a tulip screw of claim 33, comprising
an adaptor set screw for retaining the first spinal rod within the rotatably adjustable channel;
a tulip set screw for retaining the second spinal rod within the tulip.

39. The rotatably adjustable adaptor for engaging a tulip screw of claim 33, comprising an engagement feature extending from the yoke to engage the second spinal rod within the tulip.

40. The rotatably adjustable adaptor for engaging a tulip screw of claim 39, wherein the engagement feature is integral to the yoke or fixedly coupled to the yoke.

41. A method of connecting an adaptor to a tulip, comprising:
contacting the tulip with the adaptor, wherein a first spinal rod is positioned within the tulip and the adaptor comprises;
a yoke,
an extension extending radially outward relative to the tulip, defining a first portion of a joint, and connecting the yoke to,
a rotatably adjustable channel, comprising a second portion of the joint;
adjusting the rotatably adjustable channel relative the tulip;
positioning a second spinal rod within the rotatably adjustable channel; and
positioning an adaptor set screw within the rotatably adjustable channel and an adaptor set screw within the tulip, thereby connecting the adaptor and the tulip.

42. The method of claim 41, wherein the yoke comprises an engagement feature for engaging the first spinal rod.

43. The method of claim 41, wherein the adaptor comprises a collar within the rotatably adjustable channel.

44. The method of claim 41, wherein the adaptor set screw includes a threaded post for receiving a lock nut.

45. The method of claim 44, comprising tightening the lock nut and securing the tulip set screw within the tulip.
